(19) United States
(12) Patent Application Publication
(10) Pub. No.: US 2010/0139654 A1
(43) Pub. Date: Jun. 10, 2010

(54) INHALER

(75) Inventors: Ralf Thoemmes, Willich (DE); Timo Von Brunn, Ingelheim am Rheim (DE); Elmar Mock, Colombier (CH)

Correspondence Address: ROBERTS MLOTKOWSKI SAFRAN & COLE, P.C.;Intellectual Property Department, P.O. Box 10064, MCLEAN, VA, 22102-8064, US

(73) Assignees: BOEHRINGER INGELHEIM INTERNATIONAL GMBH, Ingelheim am Rhein (DE); VECTURA DELIVERY DEVICES LIMITED, Chippenham, Wiltshire (GB)

(21) Appl. No.: 12/280,106

(22) PCT Filed: Feb. 19, 2007
(86) PCT No.: PCT/EP07/01411

§ 371 Date: Jan. 12, 2009

(30) Foreign Application Priority Data
Feb. 20, 2006 (EP) 06003367.7
Feb. 20, 2006 (EP) 06003368.5

(51) Int. Cl. A61M 15/00

(52) U.S. Cl. 128/203.15

(57) ABSTRACT

An inhaler for delivering a powdered inhalation formulation from a blister strip having a plurality of blister pockets. The used part of the blister strip with emptied blister pockets is coiled by a clock spring or pushed into a helical channel. This results in a simple construction, in which the used blister strip is kept separate from the part that has not yet been used.

### BACKGROUND OF THE INVENTION

1. Field of Invention

The present invention relates to an inhaler for delivering a preferably powdered inhalation formulation from a blister strip with a plurality of blister pockets, each containing one dose of the inhalable formulation.

2. Description of Related Art

The present invention relates to an inhaler for delivering a powdered inhalation formulation from a blister strip having a plurality of blister pockets each of which contains a dose of an inhalable formulation.

UK Patent Application Publication GB 2 407 042 A, corresponding International Patent Application Publication WO 2005/037353 A1 and corresponding U.S. Patent Application Publication 2007/137645 disclose an inhaler with a rolled-up blister strip. For or during inhalation, one dose of the inhalation formulation is respectively taken from a blister pocket and this blister pocket is thereby emptied. This takes place during inhalation, e.g., when a patient breathes in, in that an air stream is passed through the previously pierced or otherwise opened blister pocket, so that the inhalation formulation in the blister pocket mixes with the air and is delivered in the desired manner. The empty blister pockets are respectively released and must be disposed of.

Also disclosed is a similar inhaler, wherein the part of the blister strip with already opened and/or emptied blister pockets—this part is also shortly called “used part” in the present invention—is stored in the inhaler. This is carried out in that the blister strip forms an endless band which can be moved in a double-threaded spiral with deflection. This structure requires relatively high forces to move the blister strip on and does not allow optimum separation of the used part from the still unused part of the blister strip. Consequently, there is a need for design solutions for optimum storage of the used part.

### SUMMARY OF THE INVENTION

Therefore, a primary object of the present invention is to provide an inhaler which allows optimum storage of a used part of a blister strip with emptied blister pockets, and/or the separation of used and unused blister pockets or parts of the blister strip in order to prevent or reduce contamination.

The above object is achieved by an inhaler as described herein.

A first aspect of the present invention provides that the used part is coiled and/or pulled into a receiving chamber by spring force. In particular, this is carried out by means of a clock spring provided in the receiving chamber which acts on the free end of the used part of the blister strip. This provides a very simple and inexpensive manner of rolling up the used part in a very compact form.

The inhaler preferably has a conveying device for stepwise advancing of the blister strip in order to enable the blister pockets to be emptied one after another for the purpose of inhaling the respective dose. According to a particularly preferred further feature, the conveying device is constructed such that the blister strip can be released stepwise and moved on to the next blister pocket preferably exclusively by spring force. This simplifies the operation as—especially with an inhaler of purely mechanical construction—there is no need for the user to move the blister strip on fully from one blister pocket to the next. Rather, the user has only to release or actuate it, and this can be done, for example, with relatively little force and a very short movement in order to release the blister strip so that it can be moved on to the next blister pocket by spring force.

According to a second, independently realizable aspect of the present invention, the used part of the blister strip is not pulled into a receiving chamber but is preferably only pushed into it. This substantially simplifies the mechanisms required and in particular reduces the number of parts needed.

The receiving chamber is preferably constructed as a channel which is, in particular, of spiral or helical configuration. It preferably has a narrow channel width which, in particular, corresponds at least substantially to the radial thickness of the used part of the blister strip. Thus, forcible guiding of the used part can easily be achieved. In particular, the used part is then helically or spirally “coiled” when it is pushed into the channel.

Particularly preferably, a conveying device of the inhaler, which is provided for stepwise advancing of the blister strip, is sufficient as the sole drive and is constructed so that on the one hand it advances the as yet unused part of the blister strip containing blister pockets which have not yet been emptied and on the other hand it pushes the unused part into the receiving chamber or channel. The conveying device is preferably arranged between a reservoir of the inhaler for the still unused part and the receiving chamber.

According to a third, also independently realizable aspect of the present invention, the inhaler comprises an additional device for compressing emptied blister pockets. This allows an essential reduction of the required size of the receiving space for the used part of the blister strip, because the emptied blister pockets require a significantly reduced space.

Particularly preferably, the conveying device and the receiving device can be actuated simultaneously or one after the other, in particular, by means of a shared actuating element, for example, by the swiveling of a lever. In particular, the additional device is formed by the conveying device.

According to a fourth, independently realizable aspect of the present invention, the receiving device is constructed such that the used part of the blister strip is coiled or bent in the same direction in which the still unused part of the blister strip, in particular, in a reservoir, is coiled or bent. This in turn enables the used part of the blister strip to be coiled or bent in a particularly compact, and thus, space-saving manner, so that the size of the receiving chamber and the size of the inhaler can be minimised.

According to a preferred further embodiment, the plane of winding of the unused part of the blister strip and the plane of winding of the used part of the blister strip are in the same plane. In this case, the reservoir and the receiving chamber are arranged side by side. This, in particular, makes it possible to minimise the height of the inhaler or to make it particularly flat in design.

According to an alternative embodiment, the winding plane of the unused part and the winding plane of the used part are located one above the other. In this case the reservoir and the receiving chamber are arranged one above the other. In particular, this minimises the area of the inhaler.

According to a fifth, independently realizable aspect of the present invention, the receiving chamber for the used part of the blister strip and the reservoir for the unused part of the blister strip are separated from one another or kept separate from one another, in particular, so that any residual inhalation formulation potentially still present in the opened and emptied blister pockets cannot enter the unused part of the blister strip—at least during normal use of the inhaler—and become deposited on the outside thereof, for example, in an undesirable manner. This could namely lead to imprecise dosage, which can be prevented by the proposed separation.

Further aspects, features, properties and advantages of the present invention will become apparent from the following description of preferred embodiments with reference to the accompanying drawings.

### BRIEF DESCRIPTION OF THE DRAWINGS

FIG. 1 is a schematic view of a proposed inhaler according to a first embodiment in the open state with a blister strip which has already been completely used up;

FIG. 2 is a schematic view of a proposed inhaler according to a second embodiment in the open state with a still largely unused blister strip;

FIG. 3 is a schematic view of a proposed inhaler according to a third embodiment which is very similar to the first one;

FIG. 4 is a schematic view of a proposed inhaler according to a fourth embodiment which is very similar to the second one.

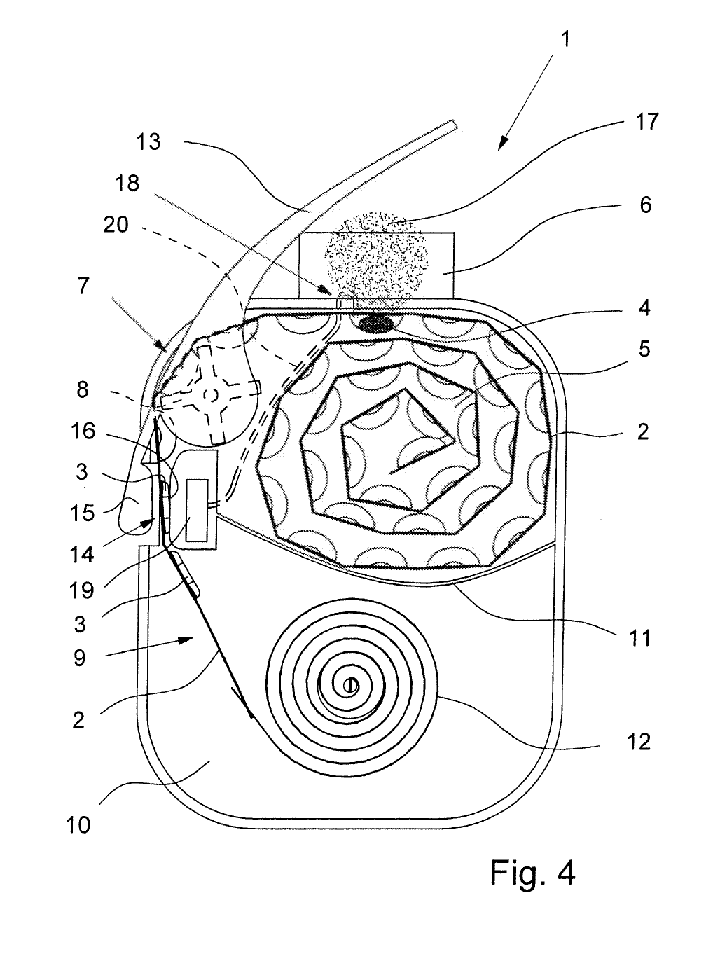

### DETAILED DESCRIPTION OF THE INVENTION

In the figures, the same reference numerals have been used for identical or similar parts, even if the associated description has not been repeated. In particular, the same or corresponding advantages and properties are achieved.

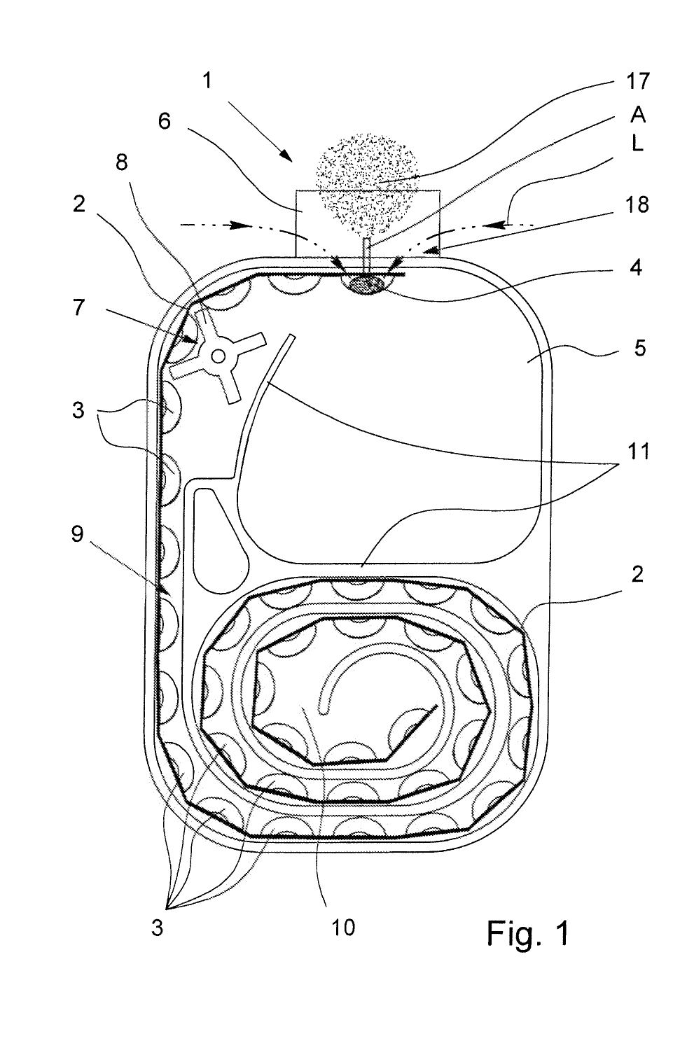

FIG. 1 shows, in highly schematic form, a proposed inhaler 1 according to a first embodiment in a cut-away or open state without a lid or cover.

The inhaler 1 serves to deliver a preferably powdered inhalation formulation from a blister strip 2 having a plurality of blister pockets 3 each of which directly contains a dose of the, in particular, loose inhalation formulation. The powder 4 that forms the inhalation formulation is shown by way of example in FIG. 1 in a blister pocket 3. For, and in particular, during inhalation, preferably one dose of the inhalation formulation is taken from a blister pocket 3.

The blister strip 2 is preferably in the form of a band or tape. Preferably, the blister strip 2 is of a finite construction, i.e., it is not in the form of an endless or closed loop.

The inhaler 1 preferably has a reservoir 5 for the as yet unused blister strip 2 with blister pockets 3 which have not yet been emptied. In particular, the blister strip 3 is rolled up or coiled in the reservoir 5. In the embodiment shown, the reservoir 5 is constructed so that the blister strip 2 can be moved or pulled out as easily as possible. In particular, there are no partition walls or inner guides in the illustrated embodiment, but rather the reservoir 5 is bounded only by preferably continuous sidewalls and flat sides. The plane of coiling or bending of the unused blister strip 2—i.e., the blister strip 2 in the reservoir 5—corresponds here to the plane of the drawing or a plane parallel thereto.

In the embodiment shown, the blister strip 2 is held directly in the reservoir 5. However, it would also be possible for a cassette, container, drum or the like containing the blister strip 2 to be inserted in the inhaler 1 or reservoir 5 instead.

The inhaler 1 has a mouthpiece 6 for a user, not shown. The individual emptying of the blister pockets 3 is carried out by means of a removal device 18, preferably with a piercing element A.

The removal device 18 is shown solely schematically here and is preferably arranged adjacent to the mouthpiece 6.

By means of the removal device 18, it is possible to open the respective blister pocket 3, for example, by piercing or cutting. In particular, using the removal device 18, the blister pocket 3 in question can be opened from the outside by being pierced or cut open by the piercing element A.

Preferably during inhalation, the opened blister pocket 3 is emptied by suction. A current L of ambient air is sucked in and is guided by the removal device 18 through the opened blister pocket 3 in such a way that the loose inhalation formulation is dispensed with the sucked-in ambient air as an aerosol cloud 17.

The inhaler 1 has a conveying device 7 for stepwise advancing of the blister strip 2, preferably, by one blister pocket 3 each time, in order to be able to feed the blister pockets 3 one after another to the removal device 18 for emptying and inhaling the respective dose.

The blister strip 2 is preferably deflected in the conveying device 7 through at most 90° in the direction of travel. This assists the desired ease of movement.

In the embodiment shown the conveying device 7 has a drive wheel 8 which can engage between the blister pockets 3, for example, and thus, advances the blister strip 2 by interlocking engagement. The conveying device 7 is preferably operated manually. Possible constructional details follow with the description of the second embodiment.

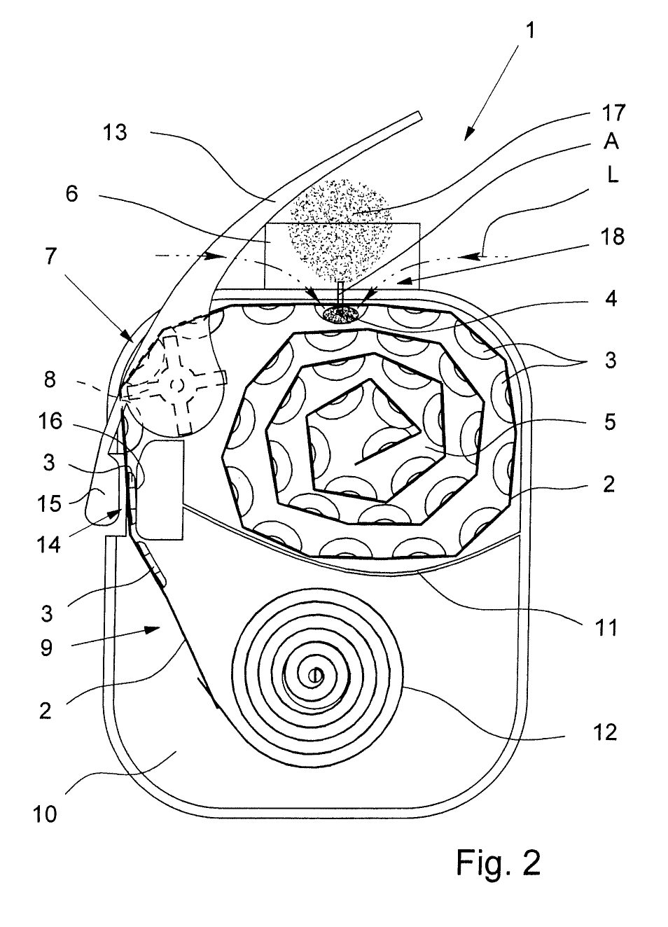

In the first embodiment, the conveying device 7 is preferably constructed such that an actuating element 13 (as shown in FIG. 2 relative to the second embodiment), particularly a cover or a housing part or the like, has to be actuated, shifted or swiveled by a user (not shown) in order to rotate the drive wheel 8 stepwise, and thereby, advance the blister strip 2 by one step.

The drive wheel 8 or the conveying device 7 is preferably constructed with a freewheel clutch and a corresponding rotation lock so that during the movement back and forth, and possibly in the event of incomplete movement of the actuating element 13, the drive wheel 8 can be rotated as desired, only in one direction, and in particular, only in the desired steps.

In the embodiment shown, the actuating element 13 can be moved in translation and/or swiveled. The movement is transmitted by means of a transmission element 15, a gear or the like, preferably, to a gearwheel 16 or the like associated with the drive wheel 8, in order to drive the drive wheel 8 in the desired manner, i.e., to advance the blister strip 2.

The inhaler 1 has a receiving device 9, particularly with a receiving chamber 10, for receiving or storing the used part of the blister strip 2.

In the first embodiment, the receiving device 9 is constructed such that, after use, i.e., after the individual blister pockets 3 have been emptied, the blister strip 2 can be pushed into the receiving chamber 10, and in particular, the blister strip 2 or the used part is accommodated in a defined and compact manner. For this purpose, the receiving chamber 10 may, for example, be provided with a guide or the like (not shown). However, the receiving chamber 10 is particularly preferably provided with a channel 14 or constructed as a channel 14 into which the blister strip 2 can be pushed.

FIG. 1 shows the inhaler 1 after repeated use and corresponding emptying of the blister pockets 3. The blister strip 2 has already been fully discharged from the reservoir 5, in the position shown, and at least the majority of it has been received by the receiving device 9 or its receiving chamber 10, i.e., pushed into the channel 14 in the embodiment shown by way of example.

The channel 14 preferably has the narrowest possible channel width which at least substantially corresponds to the radial thickness of the used part of the blister strip 2.

The channel 14 preferably extends at least substantially spirally or helically. Theoretically, other shapes are also possible, however; for example, the channel 14 may meander and/or extend in a different plane.

The receiving device 9, and particularly the inhaler 1, are preferably constructed such that the used part of the blister strip 2 is coiled or bent in the same direction in which the as yet unused part of the blister strip 2 with not yet emptied blister pockets 3 is received, particularly coiled or bent, in the inhaler 1, particularly the reservoir 5. The uniform direction of coiling or bending applies also to other areas in which the blister strip 2 is guided within the inhaler 1, in particular, i.e., for example, from the reservoir 5 to the receiving chamber 10, i.e., to the blister strip 2 in general. Because of the uniform direction of bending or coiling of the blister strip 2, it is easier to bend or coil the blister strip 2—i.e., the used part—as tightly and compactly as possible, i.e., to make the receiving chamber 10 as small and space-saving as possible. In addition, substantially lower conveying forces are needed, and consequently, the inhaler 1 is easy to operate when the blister strip 2 has to be bent around a certain radius and has not previously been bent in the opposite direction.

In the embodiment shown, the conveying device 7 is sufficiently strongly dimensioned to push the used part of the blister strip 2 into the channel. In particular, the blister strip 2 is thus moved onward or forward exclusively by the conveying device 7. In particular, the inhaler 1 has only a single conveying device 7. This results in a simple and hence inexpensive construction of the inhaler 1 which comprises only a few components.

The conveying device 7 is preferably arranged between the reservoir 5 and the receiving device 9, particularly between the removal device and the receiving chamber 10, i.e., after the emptying of the blister pockets 3.

The receiving chamber 10 is preferably separated from the reservoir 5, in this embodiment by the continuous intermediate wall 11, in particular. In this way, it is possible to prevent or at least minimize any residual inhalation formulation from falling out of the emptied and opened blister pockets 3 and accumulating on the outside of the blister strip 2 in the region of the unused part, i.e., on blister pockets 3 which are still full. The separation of the receiving chamber 10 prevents or at least minimizes possible contamination or incorrect dosing caused by these residues.

A second embodiment of the proposed inhaler 1 will now be described in more detail with reference to FIG. 2, which corresponds at least substantially to the diagram in FIG. 1. To avoid repetition, only the essential differences between the second embodiment and the first embodiment will be described hereinafter. The remarks and explanations made in relation to the first embodiment and to the present invention in general thus still apply in a corresponding or supplementary fashion.

In the second embodiment the inhaler 1 or the receiving device 9 is constructed such that the used part of the blister strip 2 is coiled and/or pulled into the receiving chamber 10 by spring force. For this purpose, the conveying device 9 preferably has a spring, particularly a clock spring 12, which is shown purely diagrammatically in its tensioned state in FIG. 2.

In the embodiment shown, the spring is arranged in the receiving chamber 10 and preferably acts on the free end of the used part of the blister strip 2. For example, the spring 2 is hooked onto the end portion of the blister strip 2.

The further forward the blister strip 2 is moved by the conveying device 7, the longer becomes the (used) part of the blister strip 2 that extends into the receiving chamber 10 and is then correspondingly coiled by the clock spring 12. However, other design solutions which have the same or similar results are also possible.

According to a particularly preferred further feature, the tensioning or pulling force of the spring, i.e., the spring force, is sufficiently great to move the blister strip 2 forward and also to pull it out of the reservoir 5, i.e., depending on the design to uncoil it as well. In this case, the conveying device 7 is preferably constructed such that the blister strip 2 can be released stepwise and is preferably advanced solely by the spring force to the next blister pocket 3. This makes the operation and handling of the inhaler 1 particularly easy as the user (not shown) then has only to operate a button (not shown) for release or unlocking. This can rule out, in particular, an incomplete advancing of the blister strip 2 from one blister pocket 3 to the next blister pocket 3 caused by improper actuation.

In the illustrated embodiment, however, the conveying device 7 is preferably designed such that an actuator element, in particular, a hand lever 13, has to be pivoted by a user to stepwise rotate the drive wheel 8 further, and, thus, move the blister strip 2 forward by one step. The drive wheel 8 or conveying device 7 is preferably provided with a freewheel and a respective rotation lock, so that the drive wheel 8 is rotatable only in one direction, and in particular, only in the desired steps by forward and back pivoting and also by incomplete forward and back pivoting of the hand lever 13.

In the second embodiment, the inhaler 1 preferably comprises an additional device 14 for compressing emptied blister pockets 3. The additional device 14 allows compressing of emptied blister pockets 3 before receipt in the receiving space 10. The compressing takes place, in particular, after the conveying device 7 or the drive wheel 8 and before the receiving space 10.

The compressing of the emptied blister pockets 3 results in an essential reduction of the required space for the used part of the blister strip 2, so that the receiving space 3 and, thus, also the inhaler 1 can be designed smaller or more compact.

Particularly preferably, the additional device 14 is coupled with the conveying device 7 or formed by it. In particular, the compression of an emptied blister pocket 3 takes place preferably directly after the onward movement of the blister strip 2 by one step, i.e., to the next blister pocket 3. More preferably, the additional device 14 is coupled with the actuating element of the conveying device 7, namely, in particular, with the hand lever 13.

In the illustrated embodiment, the additional device 14 is formed by an extension 15 of the hand lever 13, which extension causes a compression of an emptied blister pocket 3, when the hand lever 13 is completely pivoted, by clamping the respective blister pocket 3 between the extension 15, which is provided with a respective contact face, and a stationary counter contact face in the embodiment shown. Due to the respective lever transmission, a very effective compressing of emptied blister pockets 3 can be achieved. Thus, the blister strip 2 can be coiled in an essentially more compact manner by the receiving device 9—in the illustrated embodiment by the clock spring 12—than with uncompressed blister pockets 3.

A cloud 17 in FIGS. 1 and 2 schematically indicates how the inhalation formulation could be delivered during inhalation or nebulization by the inhaler 1.

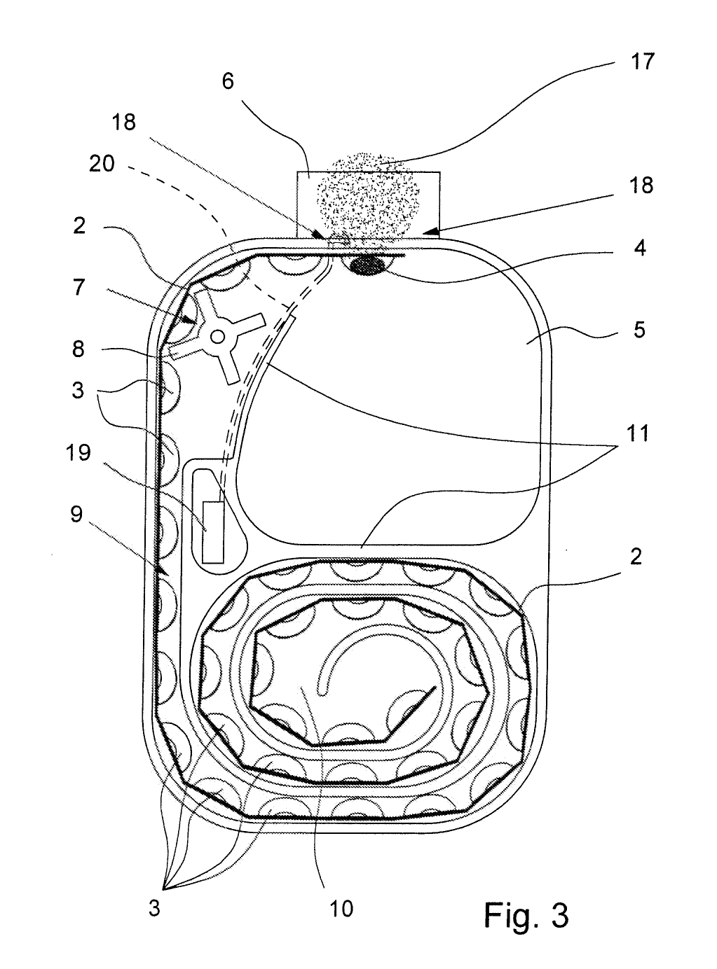

In the third and fourth embodiments shown in FIGS. 3 & 4, which largely correspond to the first and second embodiments, respectively, the inhalation formulation is expelled from the respective blister pocket 3 by means of gas or air which is under pressure. These are therefore an active inhalers 1; the preferably powdered, but possibly also liquid inhalation formulation is thus actively nebulized or expelled and not delivered by an air current generated by breathing in during the inhalation process.

The inhaler 1 or removal device 18 comprises, for this purpose, a device 19 for providing for example, ed gas. This may be, for example, a gas store for compressed and/or liquefied gas, or preferably, a manually operated air pump.

The removal device 18 comprises, for example, a feeding line 20, shown schematically, for delivering the for example, pressurized gas, particularly air, from the device 19 to the respective or opened blister pocket 3. The pressurised gas is conveyed into the blister pocket 3 in order to expel and nebulize the inhalation formulation, in particular, to form a mixture of the inhalation formulation and gas or air and thereby produce an aerosol cloud 17. However, other design solutions are also possible here as well; in particular, the inhalation formulation can be conveyed out of an opened blister pocket 3 initially along a flow path—e.g., under the effect of gravity, vibration or the like—to then be expelled and atomized by the pressurized gas.

Individual features and aspects of the embodiments and alternatives may be combined with one another as desired or used in other inhalers 1.

What is claimed is:

1-23. (canceled)

24. Inhaler for delivering an inhalation formulation from a blister strip having a plurality of blister pockets, each blister pocket containing a dose of the inhalation formulation, comprising:
a housing having a mouthpiece,
a conveying device for producing a stepwise advancing movement of the blister pockets of the blister strip to and then passed the mouthpiece,
a device for individually emptying the blister pockets for delivery of the formulation out of the mouthpiece, and
a receiving device with a receiving chamber in said housing for receiving a used part of the blister strip with the emptied blister pockets,
wherein at least one of the conveying device and the receiving device is constructed such that the used part of the blister strip is pushed into the receiving chamber during the advancing movement.

25. Inhaler according to claim 24, wherein the receiving chamber is in the form of a channel.

26. Inhaler according to claim 25, wherein the channel has a channel width which at least substantially corresponds to a thickness of the used part.

27. Inhaler according to claim 25, wherein the channel extends spirally or helically.

28. Inhaler according to claim 24, wherein the conveying device is position to act on the blister strip at a location between the receiving chamber and a reservoir in the housing for the, as yet, unused part of the blister strip with dose-containing blister pockets.

29. Inhaler according to claim 24, wherein the conveying device forms the sole drive for moving the blister strip.

30. Inhaler for delivering an inhalation formulation from a blister strip with a plurality of blister pockets, each blister pocket containing a dose of the inhalation formulation, comprising:
a housing having a mouthpiece,
a conveying device for producing a stepwise advancing movement of the blister pockets of the blister strip to and then passed the mouthpiece,
a device for individually emptying the blister pockets for delivery of the formulation out of the mouthpiece, and
a receiving device with a receiving chamber in said housing for receiving a used part of the blister strip with emptied blister pockets,
wherein the receiving device is constructed such that the used part is at least one of coiled and pulled into the receiving chamber by spring force.

31. Inhaler according to claim 30, wherein the receiving device comprises a clock spring.

32. Inhaler according to claim 31, wherein the spring is arranged in the receiving chamber.

33. Inhaler according to claim 31, wherein the spring acts on a free end of the used part.

34. Inhaler according to one of claims 30, wherein the conveying device is constructed such that the blister strip is released stepwise and advanced stepwise solely by spring force.

35. Inhaler for delivering an inhalation formulation from a blister strip with a plurality of blister pockets, each blister pocket containing a dose of the inhalation formulation, comprising:
a housing having a mouthpiece,
a conveying device for producing a stepwise advancing movement of the blister pockets of the blister strip to and then passed the mouthpiece,
a device for individually emptying the blister pockets for delivery of the formulation out of the mouthpiece,
a receiving device with a receiving chamber in said housing for receiving a used part of the blister strip with emptied blister pockets, and
a device for compressing emptied blister pockets.

36. Inhaler according to claim 35, wherein the device for compressing compresses emptied blister pockets before the respective receipt thereof in the receiving chamber.

37. Inhaler according to claim 35, wherein the device for compressing and the conveying device are operable by means of a pivoting a hand lever, the device for compressing being formed by or integrated into the conveying device.

38. Inhaler for delivering a inhalation formulation from a blister strip with a plurality of blister pockets, each blister pocket containing a dose of the inhalation formulation, comprising:
a housing having a mouthpiece,
a conveying device for producing a stepwise advancing movement of the blister pockets of the blister strip to and then passed the mouthpiece,
a device for individually emptying the blister pockets for delivery of the formulation out of the mouthpiece, and
a receiving device with a receiving chamber in said housing for receiving a used part of the blister strip with emptied blister pockets,
wherein the receiving device is constructed such that the used part is coiled or bent in the same direction in which the, as yet, unused part of the blister strip with dose-containing blister pockets is coiled or bent within a reservoir of the housing.

39. Inhaler according to claim 38, wherein an unwinding plane of the unused part and a winding plane of the used part are in the same plane.

40. Inhaler according to claim 38, wherein an unwinding plane of the unused part and a winding plane of the used part lie one above the other.

41. Inhaler for delivering an inhalation formulation from a blister strip with a plurality of blister pockets, each of the blister pockets containing a dose of the inhalation formulation, comprising:
a housing with a mouthpiece,
a reservoir in the housing for an, as yet, unused part of the blister strip with dose-containing blister pockets,
a conveying device for producing a stepwise advancing movement of the blister pockets of the blister strip to and then passed the mouthpiece,
a dispensing device for individually emptying the blister pockets for delivery of the formulation out of the mouthpiece, and
a receiving device with a receiving chamber in said housing for receiving a used part of the blister strip with emptied blister pockets,
wherein the receiving chamber is separated from the reservoir.

42. Inhaler according to claim 24, wherein the inhaler is constructed to be portable.

43. Inhaler according to claim 24, wherein the dispensing device is an air pump is constructed for individually emptying the blister pockets for delivering a respective dose by means of pressurized air.

44. Inhaler according to claim 24, wherein the blister pockets are individually openable, one after the other from the outside, so that, by breathing in while inhaling, an air current of ambient air is drawn in so as to deliver the respective dose with the ambient air as an aerosol cloud.

45. Inhaler according to claim 24, wherein the inhaler is designed to accommodate a blister strip of finite length.

46. Inhaler according to claim 24, wherein the conveying device deflects the blister strip through at most 90°.
